(19) United States
(12) Patent Application Publication
(10) Pub. No.: US 2010/0312056 A1
(43) Pub. Date: Dec. 9, 2010

(54) Endoscope shaft

(75) Inventors: Nison Galperin, Fairfield, CT (US); Lawrence James St. George, Sudbury, MA (US)

Correspondence Address: HARRINGTON & SMITH, 4 RESEARCH DRIVE, Suite 202, SHELTON, CT, 06484-6212, US

(73) Assignee: Gyrus, ACMI, Inc.

(21) Appl. No.: 12/455,642

(22) Filed: Jun. 3, 2009

(51) Int. Cl. A61B 1/008; B23P 11/00

(52) U.S. Cl. 600/141; 29/428

(57) ABSTRACT

An endoscope including a control section; and a shaft extending from the control section. The shaft includes a frame including a one-piece tube. The tube includes a plurality of slots into the tube along at least one length of the tube to form spaced sections on opposite sides of each slot. A first one of the sections comprises a projection which extends into a pocket of a second one of the sections such that the projection and pocket form an over-travel limiter to limit relative motion of the first and second sections relative to each other in at least one direction.

### BACKGROUND OF THE INVENTION

1. Field of the Invention

The invention relates to an endoscope and, more particularly, to a shaft of an endoscope.

2. Brief Description of Prior Developments

U.S. Pat. No. 6,749,560 B1, which is hereby incorporated by reference in its entirety, discloses a endoscope shaft having a tube comprises of a superelastic material and straight slots. U.S. Pat. No. 6,485,411 B1, which is hereby incorporated by reference in its entirety, discloses an endoscope shaft having a tube comprised of a superelastic material and a single spiral slot.

### SUMMARY

The following summary is merely intended to be exemplary. The summary is not intended to limit the scope of the claimed invention.

In accordance with one aspect of the invention, an endoscope is provided including a control section; and a shaft extending from the control section. The shaft includes a frame including a one-piece tube. The tube includes a plurality of slots into the tube along at least one length of the tube to form spaced sections on opposite sides of each slot. A first one of the sections comprises a projection which extends into a pocket of a second one of the sections such that the projection and pocket form an over-travel limiter to limit relative motion of the first and second sections relative to each other in at least one direction.

In accordance with another aspect of the invention, an endoscope shaft frame member is provided comprising a one-piece tube comprised of a superelastic alloy. The tube comprises a plurality of slots into the tube along at least one section of the tube. Each slot has a non-straight shape to form a projection which extends into a pocket such that the projection and pocket form an over-travel limiter to limit axial twist deformation of the tube.

In accordance with another aspect of the invention, a method is provided comprising providing a tube of superelastic alloy; and making a plurality of slots into the tube to form at least one section of the tube with an increased flexibility, wherein the slots each have a non-straight shape to form a projection which extends into a pocket such that the projection and pocket form an over-travel limiter to limit axial twist deformation of the tube.

### BRIEF DESCRIPTION OF THE DRAWINGS

The foregoing aspects and other features of the invention are explained in the following description, taken in connection with the accompanying drawings, wherein:

FIG. 1 is a side elevational view of an endoscope incorporating features of the present invention;

FIG. 2 is a cross-sectional view of the shaft of the endoscope shown in FIG. 1;

FIG. 3 is a side elevational view of the tube used for the frame of the shaft shown in FIG. 2;

FIG. 4 is an enlarged perspective view of a portion of the tube shown in FIG. 3;

FIG. 5 is a side view of a portion of the tube shown in FIGS. 3-4 showing the tube bent;

FIG. 6 is a side view of a distal end of an alternate embodiment of an endoscope without its outer cover;

FIG. 7 is an enlarged perspective view of a portion of the distal end shown in FIG. 6;

FIG. 8 is a cross sectional illustration of an alternate embodiment of the twist limiter projection shown in FIG. 4;

FIG. 9 is a cross sectional illustration of another alternate embodiment of the twist limiter projection shown in FIG. 4;

FIG. 10 is a plan top illustration of another alternate embodiment of the twist limiter projection and pocket shown in FIG. 4;

FIG. 11 is a plan top illustration of another alternate embodiment of the twist limiter projection and pocket shown in FIG. 4; and

FIG. 12 is a plan top illustration of another alternate embodiment of the twist limiter projection and pocket shown in FIG. 4.

### DETAILED DESCRIPTION OF EMBODIMENTS

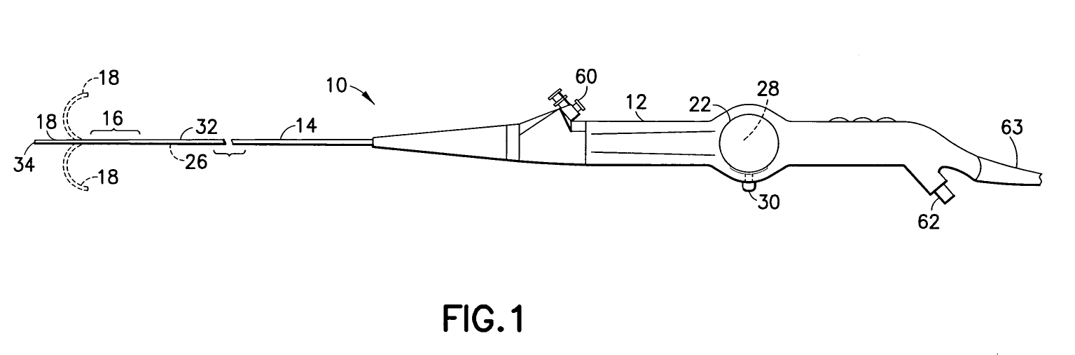

Referring to FIG. 1, there is shown a side view of an endoscope 10 incorporating features of the invention. Although the invention will be described with reference to the example embodiments shown in the drawings, it should be understood that the invention can be embodied in many alternate forms of embodiments. In addition, any suitable size, shape or type of elements or materials could be used.

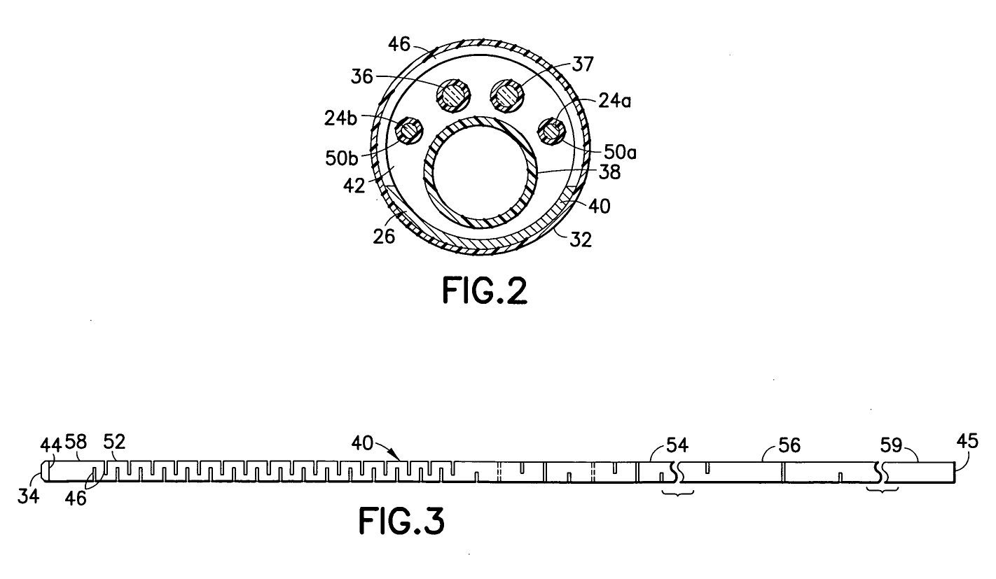

The endoscope 10 is a ureteroscope. However, in alternate embodiments the endoscope could be any suitable type of endoscope. The endoscope 10 generally comprises a handle or control 12 and a flexible or semi-flexible shaft 14 connected to the handle 12. The shaft 14 includes a passive deflection section 16 and an active deflection section 18 at the distal end of the shaft 14. A control system 22 to control the active deflection section 18 extends from the handle 12 to the active deflection section 18. Referring also FIG. 2, the control system 22 generally comprises a pair of control wires 24a, 24b, two wire sheaths 50a, 50b, and an actuator 28. The wires 24a, 24b are connected to the actuator 28 at one end and are connected to the active deflection section 18 at a second end.

In the preferred embodiment, the handle 12 has a user operated slide or lever 30. The lever 30 is connected to the actuator 28. The actuator 28 is adapted to pull and release the two wires 24a, 24b of the control system 22. When the lever 30 is moved by the user, the actuator 28 is moved. The actuator 28 may be a drum or pulley rotatably connected to the handle 12 to pull one wire 24a, 24b while releasing the other. In an alternate embodiment, the actuator may be any suitable type of device, such as a rocker arm adapted to pull and release the wires of the control system 22. In another alternate embodiment, where the control system may have two or more pairs of control wires, the handle will have additional actuators and corresponding controls to drive the additional pairs of control wires. In still other alternate embodiments, the handle may have knobs with rack and pinion mechanisms or other suitable user operated controls for the control system.

The shaft 14 is cantilevered from the handle 12. The flexible shaft 14 includes the control wires 24a, 24b of the control system 22, a fiber optical image bundle 37, a fiber optical illumination bundle 36, and a working channel 38. A port 60 for inserting instruments (not shown) into the channel 38 is located on the handle 12. The handle 12 also has a light source post 62 for connecting a light source (not shown) to the illumination bundle 36. In addition, the handle 12 has an electrical cable 63 for connection to another device, such as a video monitor. In an alternate embodiment, instead of the cable 63, the endoscope could have an eyepiece. In alternate embodiments, the flexible shaft may house different systems within.

The shaft 14 generally comprises a frame 26, a cover 32 and an objective head 34. Referring also to FIG. 3, the frame 26 generally comprises a one-piece tube 40. However, in alternate embodiments the frame could be comprised of more than one tube, such as multiple tubes connected in series, and could comprise additional members. The tube 40 is preferably comprised of a shape memory alloy material, such as Tinel or Nitinol. The shape memory alloy material is used for its superelastic properties exhibited by the material's ability to deflect and resiliently return to its natural or predetermined position even when material strains approach 4%, or an order of magnitude greater than the typical yield strain of 0.4% giving rise to plastic deformation in common metals. Thus, the term “superelastic alloy” is used to denote this type of material. The wire sheaths 50a, 50b may also be comprised of this type of material such as disclosed in U.S. Pat. No. 5,938,588 which is hereby incorporated by reference in its entirety. In an alternate embodiment the tube might not be comprised of a superelastic alloy.

The tube 40 has a center channel 42 with open front and rear ends 44, 45, and slots 46 along at least part of its length. In this embodiment the slots 46 extend more than half way through the tube. However, in alternate embodiments one or more of the slots might not extend more than half way through the tube. In this embodiment the slots have different patterns along different sections or lengths of the tube. More specifically, in this embodiment the slots 46 are configured into three sections 52, 54, 56. Each section has a different pattern of the slots 46. The pattern(s) of the slots 46 can be configured based upon, for example, the following variables:
distance or spacing between adjacent slots;
direction(s) of the slots into the tube 40;
depth of the slots into the tube;
width of the slots;
shape of the slots; and
intermixing of different directions of the slots along a length of the tube.

In alternate embodiments the tube 40 could have more or less than three sections of different slot patterns, such as only one or two for example. In addition, rather than abrupt transitions between sections of different slot patterns, the tube could be provided with gradual or intermixed slot transition zones between sections. In this embodiment the tube 40 also has two sections 58, 59 which do not have slots therein.

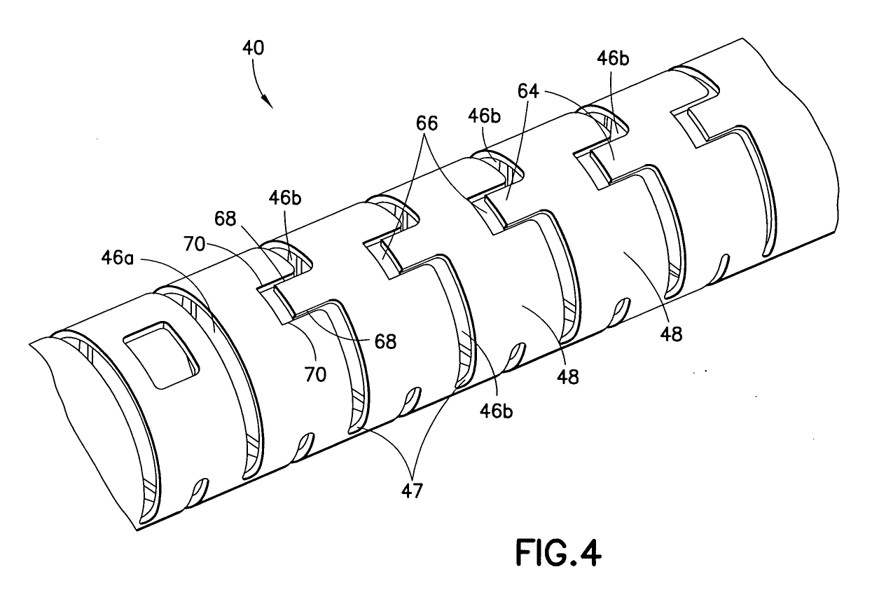

Referring also to FIG. 4, an enlarged view of a front end of the tube 40 is shown. The slots 46 include first slots 46a and second slots 46b. The first slots 46a are substantially straight, and extend into the tube generally perpendicular to the center longitudinal axis of the tube 40. The second slots 46b have a non-straight shape. In this example embodiment the second slots 46b have a general three dimensional curved general zigzag shape. This shape forms projections 64 and pockets 66. The slots form spaced sections 48 on opposite sides of each slot 46b, wherein a first one of the sections comprises one of the projections 64 which extends into the pocket 66 of an opposite second one of the sections 48. Each second slot 46b has opposite ends 47 on opposite sides of the tube which are aligned and generally perpendicular to a center axis of the tube. The first slots 46a, because they are straight, do not have the pockets and projections.

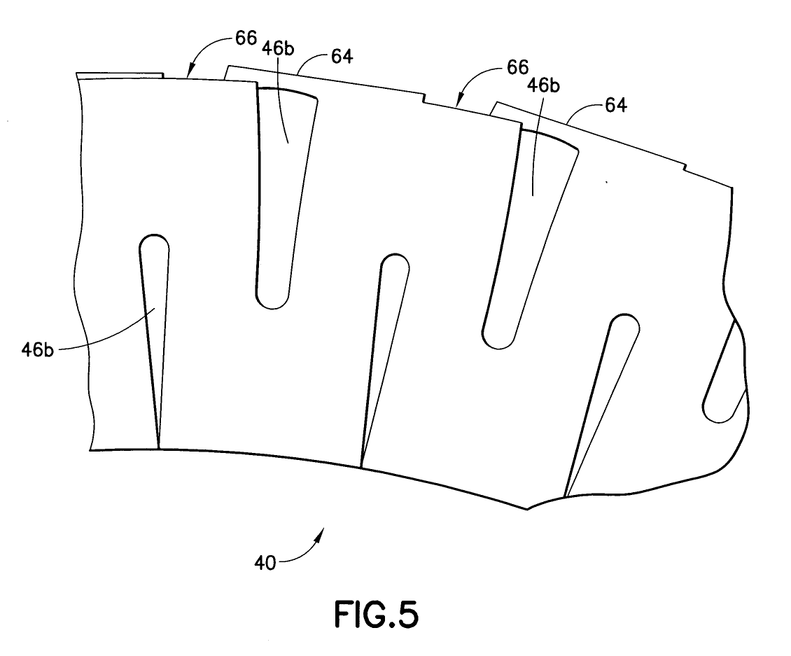

Referring also to FIG. 5, the slots 46 allow the tube 40 to bend. The projections 64 can longitudinally slide forward and backward in the pockets 66 during this bending. Lateral sides 68 of the projections 64 are normally slightly spaced from lateral sides 70 of the pockets 66. However, if the tube 40 encounters an axial torque or twisting force, the sides 68, 70 can contact each other and limit twisting of the adjacent sections 48 relative to each other. Thus, the projections and pockets form an over-travel limiter to limit relative motion of the first and second sections relative to each other in at least one direction. In this particular example the limiter limits axial twisting or deformation of the tube 40.

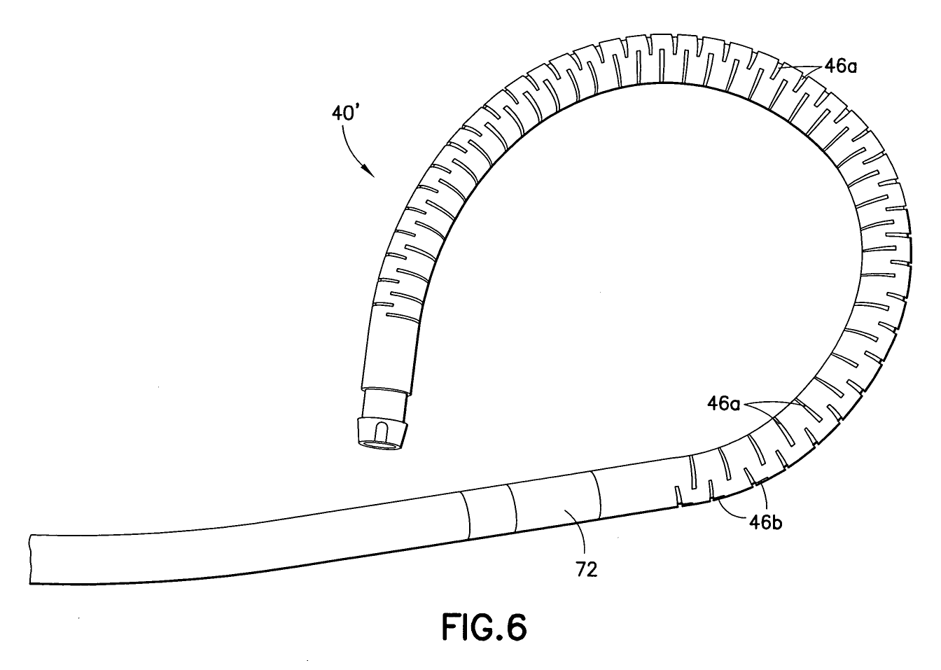

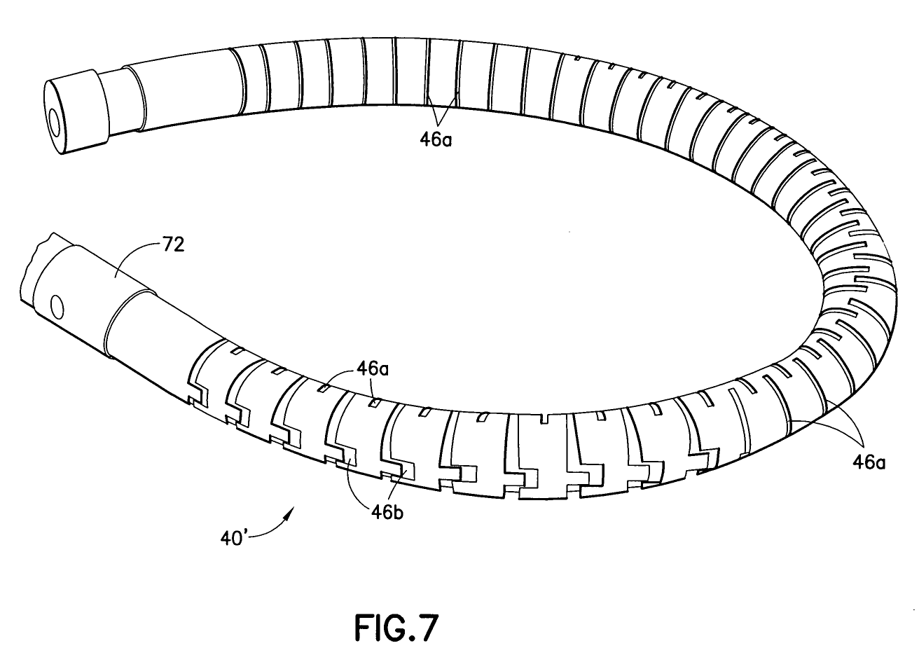

FIGS. 6 and 7 shown an alternate embodiment of the invention wherein the tube 40′ is provided only at the distal end of the shaft (the outer cover of the shaft is not shown merely for the sake of understanding). In this example embodiment the second slots 46b are merely provided at a rear section of the tube 40′ proximate a junction 72 with the rest of the shaft. In addition, the second slots 46b are merely provided at one side of the tube 40′. The first slots 46a are on the other side of the tube, interleaved with the second slots 46b, and located in front of the second slots 46b on the same side. Any suitable arrangement of the first and second slots 46a, 46b relative to each other could be provided. Additional differently shaped slots could also be provided, or the tube might only have the second slots 46b.

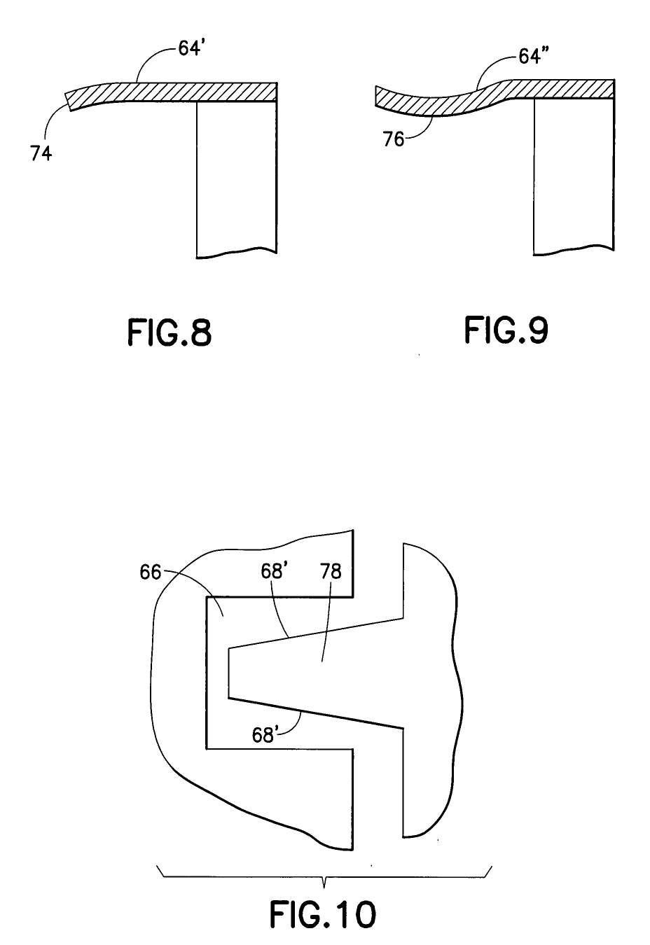

FIG. 4 shows the projection 64 as a general cantilevered rectangular shape. However, one or more of the projections 64 could have a different shape. FIG. 8 illustrates a projection 64′ with an inwardly shaped tip 74. FIG. 9 illustrates a projection 64″ with an inwardly shaped middle 76. FIG. 10 illustrates a projection 78 in a pocket 66 wherein the projection has sloped lateral sides 68′. Depending upon the longitudinal position of the projection 78 in the pocket 66 (such as based upon the amount of bend of the tube), the amount of axial twist allowed can be varied with this embodiment.

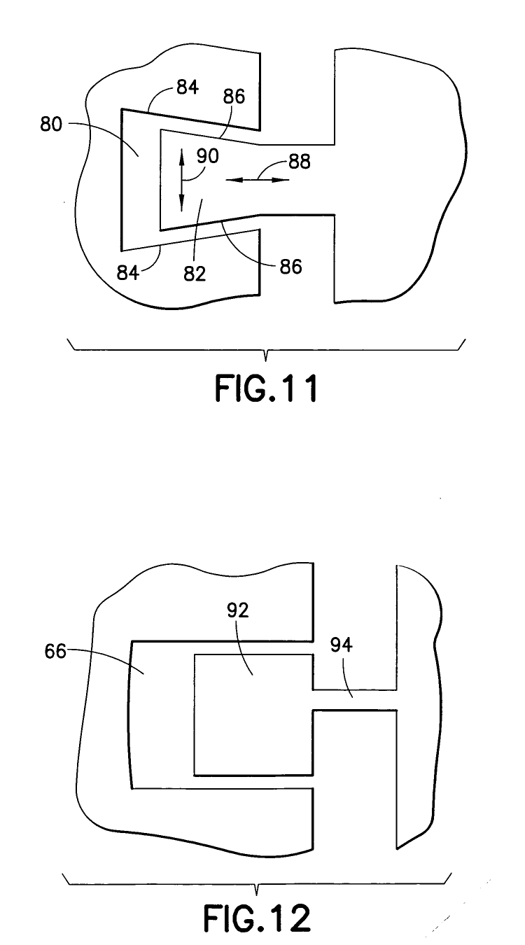

FIG. 11 illustrates another embodiment wherein the shapes of the pocket 80 and projection 82 can be used to limit longitudinal motion 88 (when the lateral sides 84, 86 wedge against each other); in addition to limiting the amount of axial twist (relative motion in direction 90). This can limit the amount of bending of the tube.

FIG. 12 illustrates another embodiment wherein the projection 92 has a resiliently deflectable spring section 94 to provide a spring action to the over-travel limiter.

With the invention, a method can be provided comprising providing a tube of superelastic alloy; and making a plurality of slots into the tube to form at least one section of the tube with an increased flexibility, wherein the slots each have a non-straight shape to form a projection which extends into a pocket and can longitudinally move relative to the pocket but has limited lateral movement in the pocket, such that the projection and pocket form an over-travel limiter to limit axial twist deformation of the tube. The method of making the slots can include, for example, laser forming of the slots in the tube.

Conventional endoscopes having a tube frame member comprising a superelastic alloy with slots perpendicular to deflections plane are known as noted above. Geometry of these slots corresponds to the requirements needed in the deflection elasticity. Slotted tubes, in some cases made from laser-cut tubing, have been used in the active deflection portion of flexible ureteroscopes with good success for a number of years. Generally, the slotted tubes have been designed to deflect in one direction, or opposing directions, and the length of the slotted tubes at maximum has been on the order of about two inches.

Newer designs of endoscopes have been using longer slotted tubes with similar defection capability in two opposing directions, but these longer version slotted tubes have shown some propensity to break at the proximal end of the tube. The present understanding is that the longer slotted tube is more likely to experience a higher torque force (than the shorter slotted tubes in earlier designs) in the proximal end as the endoscope tip at the distal end is being manipulated to the sides during a medical procedure (twisted). The earlier designs seem to have been more flexibility in the proximal end of the endoscope's deflection section, whereas deflection sections utilizing a longer slotted tube (about 3 inches long) do not have such proximal section flexibility. This stronger torque force can strongly twist and deform the proximal section of the long slotted tube and, this deformation can lead to material fatigue despite the use of superelastic material as the frame of the slotted tube. Existing slotted tube frame members work well with deflection loads, but cannot withstand angular loads (torque) because higher “deflection flexibility”, lower “torque resistance stability”.

With the longer slotted tubes noted above, the proximal end of the slotted tube (prior to the bend) seems to be absorbing the twist, with some prominent bend lines showing from the bottom of the open slots into the adjacent slots in that area, and the tube construction did not seem to allow the twist to propagate to the tip. Thus, tip steering only seemed to be possible to the extent that the whole distal end of the shaft could sweep with the shaft staying in the plane of the bend; essentially a straight line, no bending around an orthogonal corner.

One of the purposes of the invention is to reduce the deformation of the material of the proximal section of the slotted tube due to a strong twistings and, thus, eliminate a large source of material fatigue. A basic difference of the proposed design is that the rings (sections 48) between the slots have protrusions or tabs at the center of the slot, directed along the axis of the slotted tube, and associated notches on the following coil (section 48) of the tube. The protrusion or tab 64 can function as a key. The locations of the pockets 66 is perpendicular to the plane of deflection, and this should improve the durability of the slotted tube significantly. The solution can help to resolve the physical contradiction of higher deflections flexibility, and lower torque resistance stability. Implementation of the proposed slotted tube key design will not only increase the tube torque resistance, it will also make the slotted tube more stable in the deviation from bending plane (skew).

If twisted, the rings/coils in a conventional slotted tube frame member could and would shift transversely relative to each other; causing the web of material between adjacent slots to deform and perhaps creases form at sites where the tube material would experience stress. With the invention on the other hand, when the section with interlocking tabs (keys) is twisted, the tabs transfer the twisting force onto the next ring (section 48) with very little relative transverse displacement. This virtually eliminates the excessive material deformation and associated excessive stress. The tab 64 extends into the adjacent slit 66 enough so that when the slotted tube deflects there is still engagement of tab to slot. Tab (key) geometry may be varied to allow for variations in overall tube design, but a fundamental purpose is preserved; to translate the twisting force to the next ring (section 48) with a minimal amount of relative transverse displacement between existing sections 48 and, thus, a minimal amount of material deflection and associated stress.

It should be understood that the foregoing description is only illustrative of the invention. Various alternatives and modifications can be devised by those skilled in the art without departing from the invention. For example, features recited in the various dependent claims could be combined with each other in any suitable combination(s). In addition, features from different embodiments described above could be selectively combined into a new embodiment. Accordingly, the invention is intended to embrace all such alternatives, modifications and variances which fall within the scope of the appended claims.

What is claimed is:

1. An endoscope comprising:
a control section; and
a shaft extending from the control section, wherein the shaft includes a frame comprising a one-piece tube, wherein the tube comprises a plurality of slots into the tube along at least one length of the tube to form spaced sections on opposite sides of each slot, wherein a first one of the sections comprises a projection which extends into a pocket of a second one of the sections such that the projection and pocket form an over-travel limiter to limit relative motion of the first and second sections relative to each other in at least one direction.

2. An endoscope as in claim 1 wherein the tube is comprised of a superelastic alloy.

3. An endoscope as in claim 1 wherein the slots extend more than half way through the tube.

4. An endoscope as in claim 1 wherein the slots extend into the tube from two opposite sides of the tube.

5. An endoscope as in claim 1 wherein the tube further comprises a second plurality of slots which do not comprises the projections and pockets.

6. An endoscope as in claim 1 wherein the at least one direction is an axial twist direction.

7. An endoscope as in claim 1 wherein the projection is configured to slide generally longitudinally forward and backward the pocket.

8. An endoscope as in claim 1 wherein lateral sides of the projection are located to contact opposite lateral sides of the pocket when the tube is axially twisted.

9. An endoscope as in claim 1 wherein each slot has a three dimensional curved general zigzag shape.

10. An endoscope as in claim 9 wherein each slot has opposite ends on opposite sides of the tube which are aligned and generally perpendicular to a center axis of the tube.

11. An endoscope shaft frame member comprising a one-piece tube comprised of a superelastic alloy, wherein the tube comprises a plurality of slots into the tube along at least one section of the tube, wherein each slot has a non-straight shape to form a projection which extends into a pocket such that the projection and pocket form an over-travel limiter to limit axial twist deformation of the tube.

12. An endoscope shaft frame member as in claim 11 wherein the slots extend more than half way through the tube.

13. An endoscope shaft frame member as in claim 11 wherein the slots extend into the tube from two opposite sides of the tube.

14. An endoscope shaft frame member as in claim 11 wherein the tube further comprises a second plurality of slots which do not comprises the projections and pockets.

15. An endoscope shaft frame member as in claim 11 wherein the projection is configured to slide generally longitudinally in an arc forward and backward in the pocket.

16. An endoscope shaft frame member as in claim 11 wherein lateral sides of the projection are located to contact opposite lateral sides of the pocket when the tube is axially twisted.

17. An endoscope shaft frame member as in claim 11 wherein each slot has a three dimensional curved general zigzag shape.

18. An endoscope shaft frame member as in claim 11 wherein each slot has opposite ends on opposite sides of the tube which are aligned and generally perpendicular to a center axis of the tube.

19. An endoscope comprising:
a control section; and
a shaft extending from the control section, wherein the shaft includes a frame comprising an endoscope shaft frame member as in claim 11.

20 A method comprising:
providing a tube of superelastic alloy;
making a plurality of slots into the tube to form at least one section of the tube with an increased flexibility, wherein the slots each have a non-straight shape to form a projection which extends into a pocket such that the projection and pocket form an over-travel limiter to limit axial twist deformation of the tube.
